(19) United States
(12) Patent Application Publication
(10) Pub. No.: US 2025/0352188 A1
(43) Pub. Date: Nov. 20, 2025

(54) AUTOMATED OSCILLATING RACK FOR URINE BAG AND METHOD AND DEVICE FOR SELF-PROPELLED PHYSIOLOGICAL FLUID COLLECTION SYSTEM

(71) Applicant: CHANGHUA CHRISTIAN MEDICAL FOUNDATION CHANGHUA CHRISTIAN HOSPITAL, Changhua City (TW)

(72) Inventors: Shao Li HAN, Changhua City (TW); Yeong Hwa CHANG, Taoyuan City (TW); Hung Wei LIN, Changhua City (TW); Yu Hsiang CHAI, Taoyuan City (TW); Yu Kai YANG, Changhua City (TW); Shin Hwar WU, Changhua City (TW); Yi Chen HUANG, Changhua City (TW); Chih Ming LIN, Changhua City (TW)

(21) Appl. No.: 19/091,654

(22) Filed: Mar. 26, 2025

(30) Foreign Application Priority Data
May 14, 2024 (TW) 113117779
Jun. 20, 2024 (TW) 113122914

(51) Int. Cl. A61B 10/00

(52) U.S. Cl. CPC A61B 10/0096; A61B 2560/0437; A61B 2562/0247

(57) ABSTRACT

An automated oscillating rack comprises a base having a first side surface attached to a support frame with a mounting space for a fluid collection bag, a primary driving unit disposed on a second side surface of the base, and an oscillating assembly comprising a main body disposed at a first end of the primary driving unit, which drives the main body between a first and a second position; a flow channel communicating with an outlet of the fluid collection bag; a secondary driving unit disposed on a second side of the main body; and a flow control switch having a first end attached to a middle section of the flow channel for opening or closing the flow channel and a second end at the secondary driving unit driving the first end to move between a third and a fourth position for opening or closing the flow channel.

### FIELD OF INVENTION

The present invention relates to a medical system designed for the collection of physiological fluids, in particular to an automated oscillating rack for urine bag and to a method and device for a self-propelled physiological fluid collection system.

### BACKGROUND OF THE INVENTION

In today's hospital environment, continuous monitoring of urine output serves as a vital metric for assessing patient health, particularly among those with critical conditions in Intensive Care Units (ICUs). Such monitoring relies on urinary catheters to provide accurate measurements of urine volume, providing critical insight into physiological function. However, the need to connect the urinary catheter to a fluid collection bag for temporary storage presents an operational challenge. As urine accumulates, the bag must be periodically or irregularly emptied by nursing staff at intervals dictated by the rate of urine production or clinical guidelines. The way to record urine output is to use the scale of the urinal (urinal) or its weight to record the amount.

Documentation of the volume of urine emptied is essential, unless explicitly exempted by medical orders. Typically, this documentation to record urine output is to use the scale of the urinal or its weight to record the amount, with cumulative data systematically entered into the hospital's electronic medical record. Although the technical complexity of this task is minimal, its execution is labor-intensive, compounded by the diverse physiological conditions of patients and the variability of emptying schedules. The resulting burden on nursing staff detracts from their ability to perform more specialized and effective nursing tasks. From an operational standpoint, the delegation of fluid collection bag management to dedicated personnel could significantly reduce this burden, allowing healthcare providers to focus on their core clinical competencies. However, existing practices constrain the optimal allocation of nursing resources and fail to address the inefficiencies inherent in manual urine management. Given that the process of emptying and documenting urine output has little impact on patient recovery outcomes, there is an urgent need to develop systems that streamline these operations while maintaining accuracy and reliability.

### SUMMARY OF THE INVENTION

A primary purpose of the present invention is to provide real-time efficiency in emptying and recording urine volume according to each patient's condition.

A secondary purpose of the present invention is to significantly reduce the nurse's workload associated with emptying fluid collection bags, allowing them more time to provide nursing care.

A further purpose of the present invention is to reduce labor costs, prevent misreading of urine volume, and ensure consistent emptying of fluid collection bags in medical care.

In order to achieve these purposes, the present invention provides an automated oscillating rack comprising a base, a primary driving unit, and an oscillating assembly. A first side surface of the base is attached to a support frame having a mounting space for accommodating a fluid collection bag. A first side of the primary driving unit is disposed on a second side surface of the base to facilitate the movement of the oscillating assembly. The oscillating assembly comprises a main body having a first side disposed at a first end of the primary driving unit, which drives the main body to move between a first position and a second position; a flow channel formed as a passage, a first end of the flow channel is in communication with an outlet of the fluid collection bag to allow a controlled flow of physiological fluid; a secondary driving unit disposed on a second side of the main body relative to the flow channel; and a flow control switch having a first end attached to the flow channel for opening or closing the passage of the flow channel and a second end disposed at a first end of the secondary driving unit, wherein the secondary driving unit drives the first end of the flow control switch to move between a third position and a fourth position, thereby causing the passage of the flow channel to be opened or closed.

The following describes several preferred embodiments of the present invention in detail with reference to the accompanying drawings.

### BRIEF DESCRIPTION OF THE DRAWINGS

FIG. 1 is a perspective view showing a preferred embodiment of the present invention attached to a support frame;

FIG. 2 is a partially exploded perspective view of FIG. 1;

FIG. 3 is a perspective view of the preferred embodiment;

FIG. 4 is another perspective view of FIG. 3 from a different angle;

FIG. 5 is an exploded perspective view of FIG. 3;

FIG. 6 is a view showing the main body at a first position and the flow control switch at a fourth position in the preferred embodiment;

FIG. 7 is a view showing the main body at a second position in the preferred embodiment;

FIG. 8 is a sectional view along direction 8-8 of FIG. 6, showing the main body at the first position and the flow control switch at the fourth position;

FIG. 9 is a view showing the main body at the first position and the flow control switch at a third position as shown in FIG. 8;

FIG. 10 is a perspective view of the self-propelled physiological fluid collection device in a preferred embodiment of the present invention;

FIG. 11 is another perspective view of FIG. 10 from a different angle;

FIG. 12 is a partially exploded view of FIG. 10;

FIG. 13 is a partially exploded view of FIG. 11;

FIG. 14 is a flowchart showing the implementation steps of the present invention; and

FIG. 15 is the flowchart continuing from FIG. 14.

### DETAILED DESCRIPTION OF THE PREFERRED EMBODIMENTS

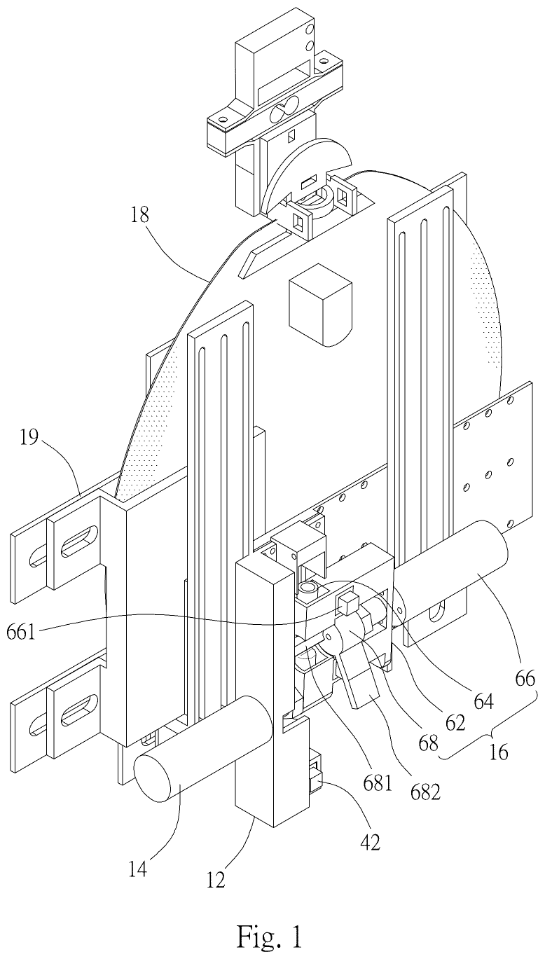

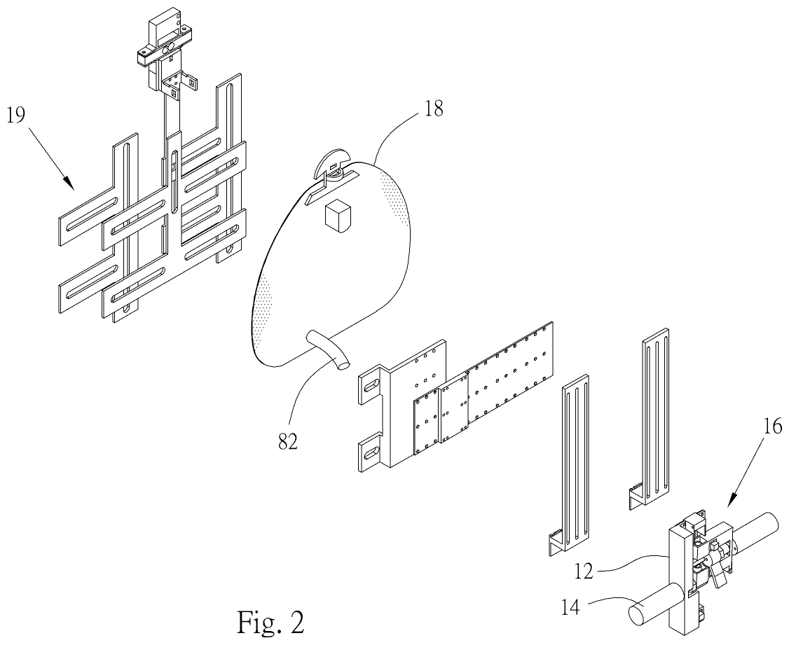

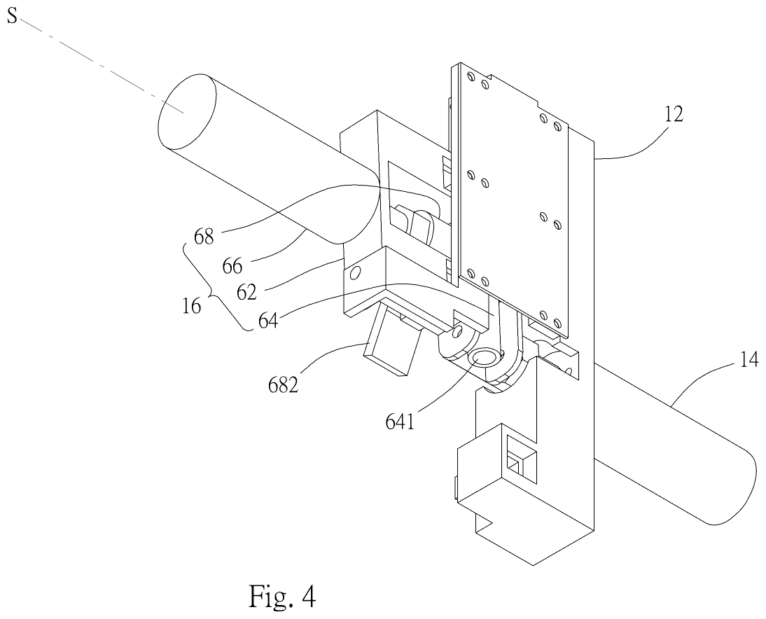

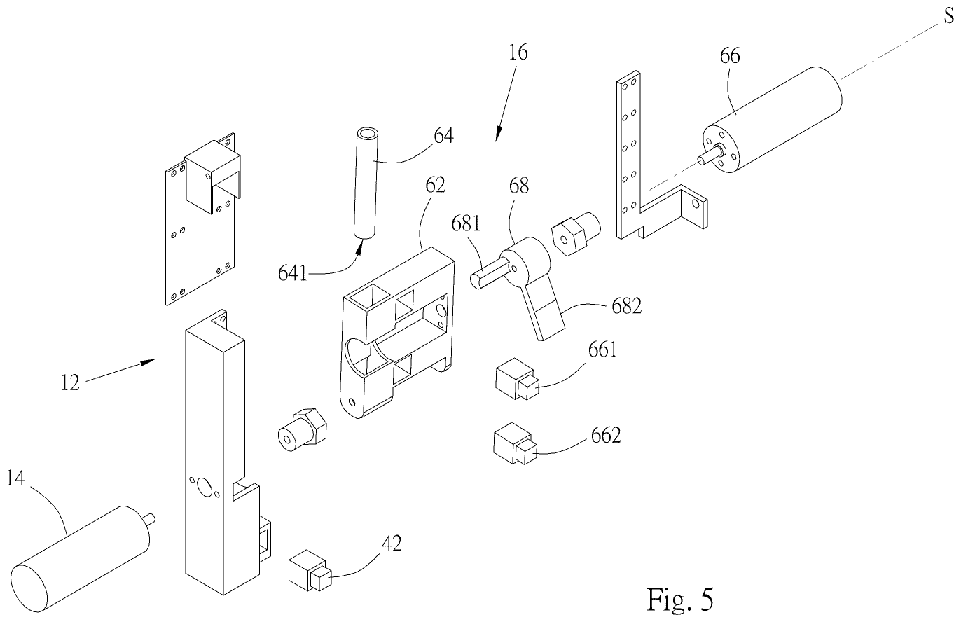

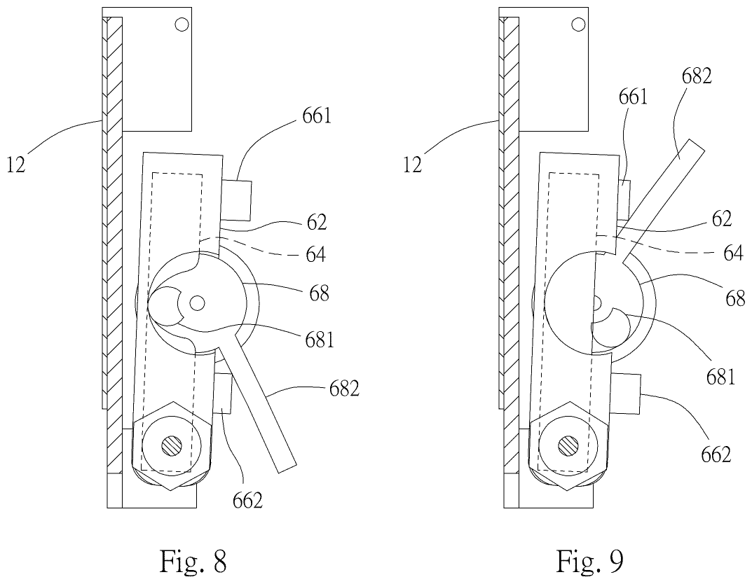

Referring to FIGS. 1 to 9, an automated oscillating rack for urine bag disclosed by the present invention comprises at least a base 12, a primary driving unit 14, and an oscillating assembly 16.

A first side surface of the base 12 is attached to a support frame 19, wherein the support frame 19 has a mounting space for accommodating a fluid collection bag 18 such as a urine bag.

A first side of the primary driving unit 14 is disposed on a second side surface of the base 12, wherein the second side surface is adjacent to the first side surface of the base 12.

The oscillating assembly 16 comprises at least a main body 62, a flow channel 64, a secondary driving unit 66, and a flow control switch 68.

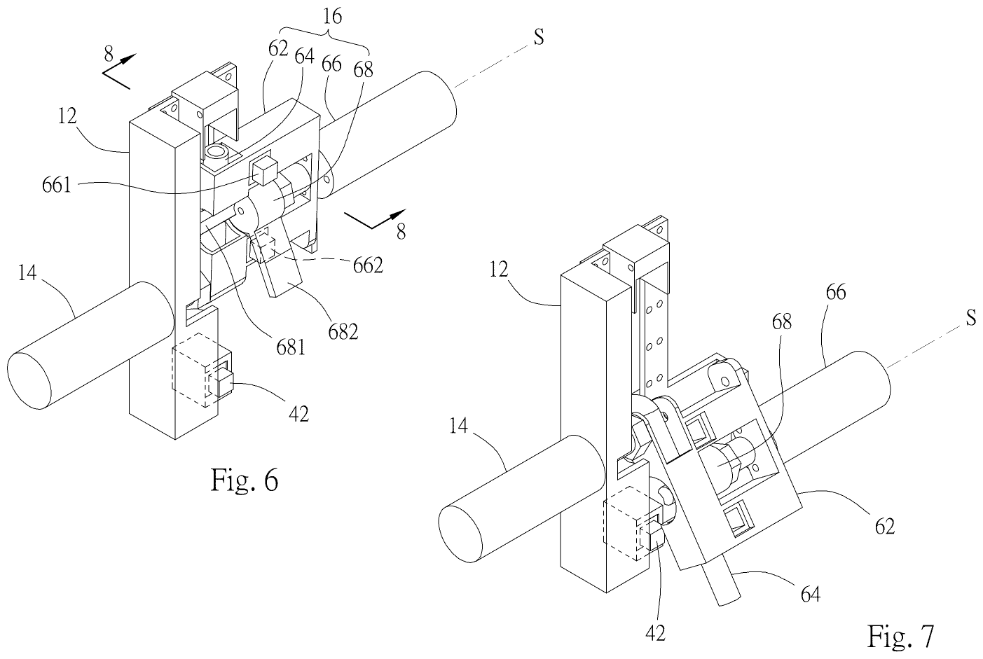

A first side of the main body 62 is disposed at a first end of the primary driving unit 14, wherein the primary driving unit 14 drives the main body 62 to move between a first position (as shown in FIG. 6) and a second position (as shown in FIG. 7).

The flow channel 64 comprises a passage, such as a flexible tube, disposed on the main body 62, wherein a first outer end 641 of the flow channel 64 communicates with an outlet 82 of the fluid collection bag 18.

The secondary driving unit 66 is disposed on a second side of the main body 62 relative to the flow channel 64.

A first end 681 of the flow control switch 68 is attached to a middle section of the flow channel 64, wherein the first end 681 of the flow control switch 68 is configured to open or close the passage of the flow channel 64.

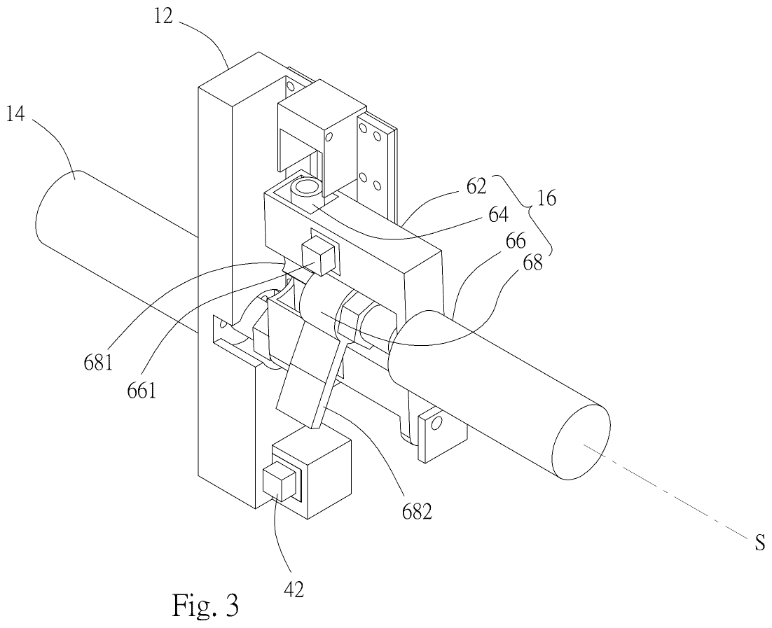

A second end of the flow control switch 68 is disposed at a first end of the secondary driving unit 66, wherein the secondary driving unit 66 drives the first end 681 of the flow control switch 68 to move between a third position (as shown in FIG. 9) and a fourth position (as shown in FIGS. 3, 6, and 8), thereby causing the passage of the flow channel 64 to be opened (as shown in FIG. 9) or closed (as shown in FIGS. 3, 6, and 8).

In the automated oscillating rack for urine bag disclosed by the present invention, the primary driving unit 14 comprises a rotary motor connected to a power source, wherein the main body 62 undergoes an angular displacement between the first position and the second position. When the main body 62 is at the first position, a second end of the flow channel 64 (i.e., the outlet) faces upward, and when the main body 62 is at the second position, the second end of the flow channel 64 faces downward to facilitate outflow of physiological fluid from the fluid collection bag 18.

In the automated oscillating rack for urine bag disclosed by the present invention, the secondary driving unit 66 comprises a rotary motor connected to a power source. The second end of the flow control switch 68 corresponds to a rotation axis S for rotation, causing the first end 681 of the flow control switch 68 to undergo an angular displacement between the third position (as shown in FIG. 9) and the fourth position (as shown in FIGS. 3, 6, and 8). The first end 681 of the flow control switch 68 comprises either a cam (not shown) or a cam block that deviates from the rotation axis S in an axial direction, such that when the cam or the cam block is at the third position, the cam or the cam block does not compress the flow channel 64 thereby opening the passage of the flow channel 64, and when the cam or the cam block is at the fourth position, the cam or the cam block compresses the flow channel 64 thereby closing the passage of the flow channel 64.

Referring again to FIGS. 1 to 9, the automated oscillating rack for urine bag disclosed by the present invention further comprises an oscillation stop button 42 disposed on the base 12 and corresponding to the second position, wherein the oscillation stop button 42 is electrically connected to the primary driving unit 14. When the primary driving unit 14 drives the main body 62 to move between the first position and the second position, the main body 62 presses the oscillation stop button 42, thereby causing the primary driving unit 14 to stop.

The automated oscillating rack for urine bag disclosed by the present invention further comprises a switch stopper 682, a first stop button 661, and a second stop button 662. The switch stopper 682 includes an extension rod extending outward from the middle section of the flow control switch 68 by a predetermined length. The first stop button 661 and the second stop button 662 are disposed on the main body 62 and positioned on opposite sides of the flow control switch 68, wherein the first stop button 661 and the second stop button 662 are electrically connected to the secondary driving unit 66 and correspond to the third position (as shown in FIG. 9) and the fourth position (as shown in FIGS. 3, 6, and 8), respectively. When the secondary driving unit 66 drives the first end 681 of the flow control switch 68 to move between the third position and the fourth position, the switch stopper 682 is configured to prevent the first stop button 661 and the second stop button 662 from being pressed simultaneously, thereby causing the secondary driving unit 66 to stop.

The automated oscillating rack for urine bag disclosed by the present invention further comprises a message transceiving unit (not shown) such as a Programmable Logic Controller (PLC), wherein the message transceiving unit is electrically connected to a command transceiving unit (not shown), the primary driving unit 14, and the secondary driving unit 66. The electrical connection between the message transceiving unit and the command transceiving unit is established through one of a wireless connection and an internet connection. When a physiological fluid collection system equipped with the command transceiving unit reaches the automated oscillating rack, the command transceiving unit notifies the message transceiving unit to instruct the primary driving unit 14 and the secondary driving unit 66 to perform an oscillating motion to complete a physiological fluid collection operation.

The message transceiving unit is configured to connect to the command transceiving unit through General Packet Radio Service (GPRS) technology with internet functionality. A Web API or Web Service API application programming interface can be established between the message transceiving unit and the command transceiving unit to process communication between a web server and a web browser.

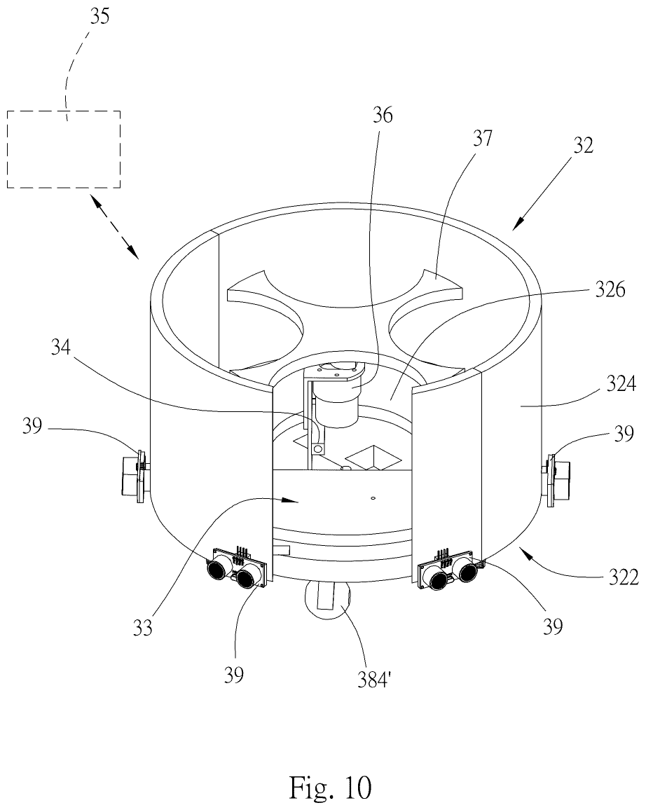

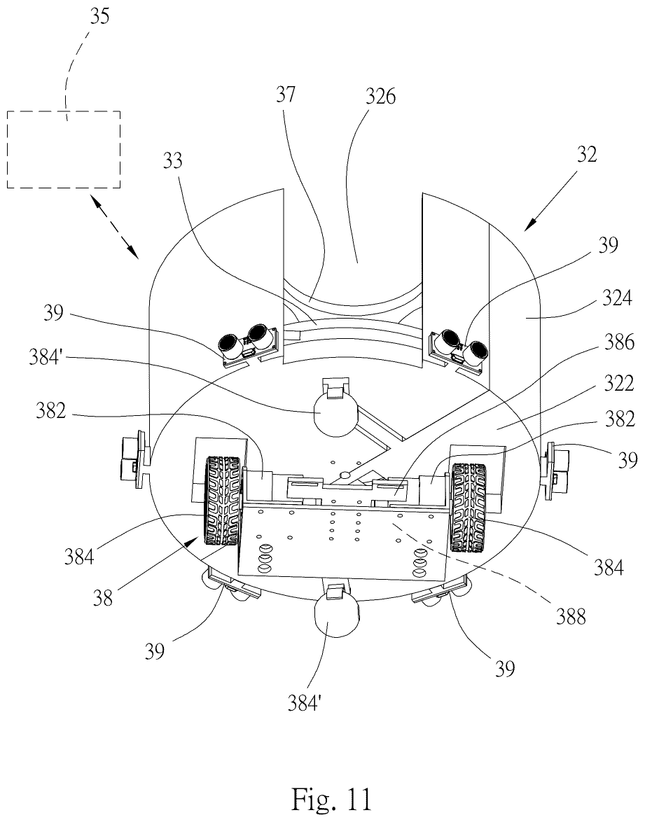

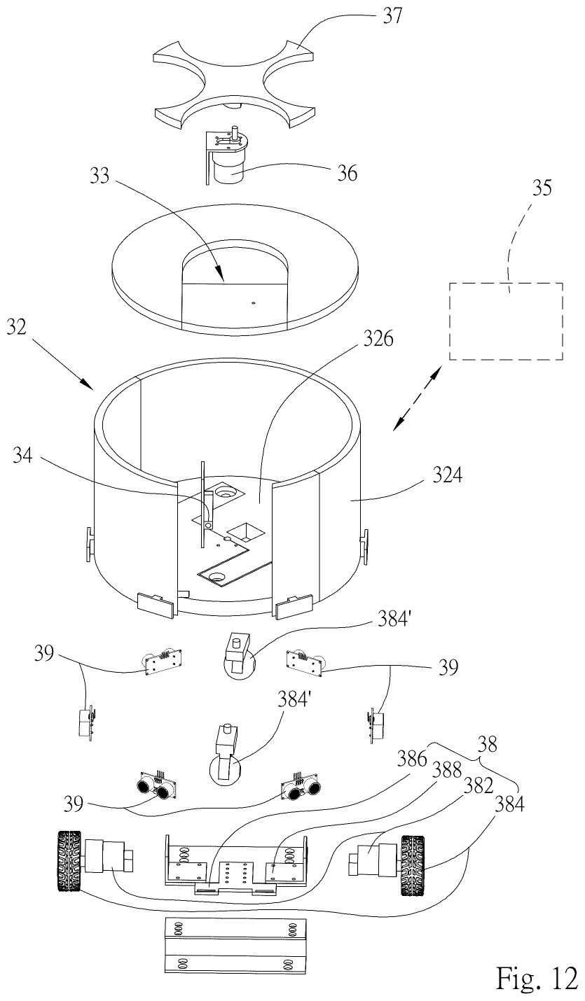

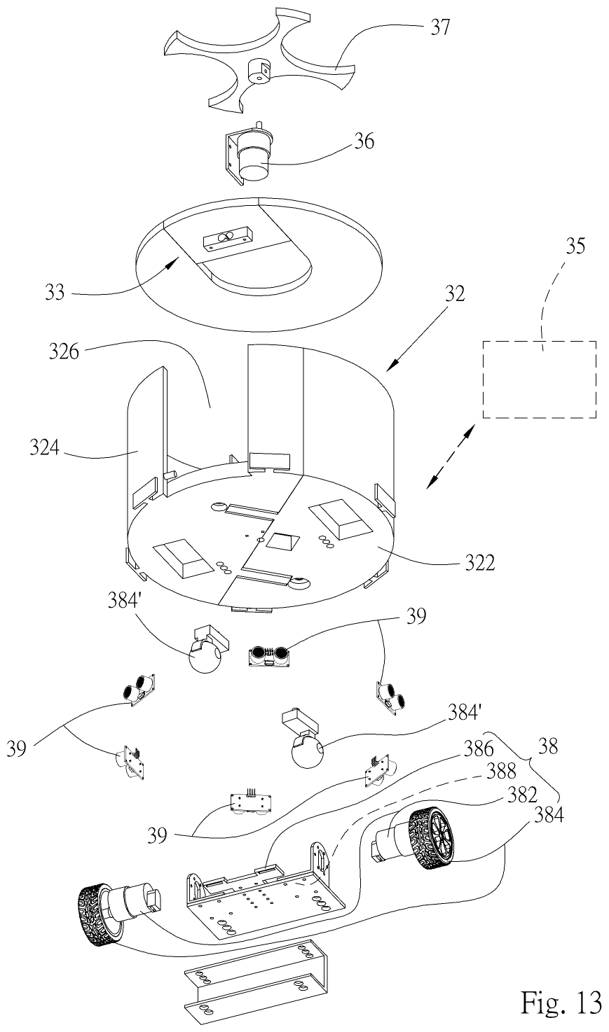

Referring to FIGS. 10 to 13, a self-propelled physiological fluid collection system of the present invention comprises a device including at least a collection unit 32, a weight sensing unit 33, a sensor 34, a primary message transceiving unit 35, a tertiary driving unit 36, a container holder 37, and a mobility driving system 38.

The collection unit 32 comprises a bottom surface 322 and a peripheral side surface 324 surrounding a periphery of the bottom surface 322, wherein the bottom surface 322 and the peripheral side surface 324 form a recessed enclosure having at least one opening 326, wherein the recessed enclosure is able to be substantially cylindrical.

A side of the weight sensing unit 33 disposes on an inner side of the bottom surface 322 and other side of the weight sensing unit 33 configured to sense a weight (not shown) placed through the opening 326, wherein the weight comprises a container or a container containing a physiological fluid, and wherein the weight sensing unit 33 is configured to transmit weight information corresponding to the sensed weight.

The sensor 34 is disposed on the collection unit 32 and configured to detect presence or absence of the container (not shown) adjacent to the weight sensing unit 33, wherein the sensor 34 is configured to transmit container presence information indicating the presence or absence of the container.

The primary message transceiving unit 35 is electrically connected to the weight sensing unit 33 and the sensor 34, wherein the primary message transceiving unit 35 is configured to receive the weight information and the container presence information and transmit a first indication message, and wherein the primary message transceiving unit 35 comprises at least one of a computer, a server, and/or a database configured for wireless communication.

The first indication message comprises at least one of a destination message, a system initialization message, and/or a system activation message, wherein the destination message indicates at least one of a hospital bed location or a fluid collection bag location.

The tertiary driving unit 36 includes a drive motor disposed on the inner side of the bottom surface 322.

A side of the container holder 37 disposes on a side of the tertiary driving unit 36, wherein the container holder 37 is configured to accommodate the container and is driven by the tertiary driving unit 36 to oscillate between a fifth position and a sixth position. When the sensor 34 detects the presence of the container and the weight sensing unit 33 detects the absence of the weight at the fifth position, the tertiary driving unit 36 maintains the container holder 37 at the fifth position. The fifth position corresponds/defines to a state where the container is present and empty, thereby being ready for a subsequent fluid collection operation.

When the sensor 34 detects presence of the container and the weight sensing unit 33 detects presence of the weight, the tertiary driving unit 36 drives the container holder 37 to the sixth position, wherein the movement to the sixth position enables the placement of another container at the fifth position for subsequent fluid collection operations.

When the sensor 34 detects absence of the container, the tertiary driving unit 36 drives the container holder 37 to the sixth position, thereby enabling another container holder 37 having a container to be positioned at the fifth position for subsequent fluid collection operations.

A side of the mobility driving system 38 disposes on an outer side of the bottom surface 322, wherein the mobility driving system 38 is electrically connected to the primary message transceiving unit 35 and configured to receive the first indication message, and wherein the mobility driving system 38 includes at least two drive motors 382, at least two main wheels 384, a navigation sensor 386, and a secondary message transceiving unit 388.

Each of the two drive motors 382 comprises a connection side connected to a respective one of the two main wheels 384, wherein the each drive motor 382 is configured to drive the each main wheel 384 to rotate for forward movement, backward movement, and/or turning movement. The mobility driving system 38 further comprises at least one auxiliary wheel 384′ configured to enhance movement stability of the device.

The navigation sensor 386 is configured to output a magnetic field message and detect a magnetic field path such as a magnetic strip disposed on a ground surface where the device is configured to follow the magnetic field path for movement operation.

The secondary message transceiving unit 388 includes at least one application installed on a circuit board, wherein the secondary message transceiving unit 388 is configured to receive the first indication message and the magnetic field message and to transmit a second indication message to the two drive motors 382 to drive the two main wheels 384 to move the collection unit 32 along the magnetic field path to a destination for collecting the weight.

In an alternative embodiment, the navigation sensor 386 is an image sensor configured to output an image message to the secondary message transceiving unit 388, which then transmits the second indication message to the two drive motors 382 to drive the two main wheels 384 to move the collection unit 32 to a destination for collecting the weight.

Referring again to FIGS. 10 to 13, the device of the self-propelled physiological fluid collection system further comprises an obstacle detection module 39 disposed on the peripheral side surface 324 of the collection unit 32, wherein the obstacle detection module 39 includes at least three spaced-apart emitters selected from the group consisting of ultrasonic emitters and infrared emitters. The obstacle detection module 39 is configured to emit waves outwardly to detect the presence or absence of an obstacle, and to transmit an obstacle presence message to the secondary message transceiving unit 388 upon detecting the presence of the obstacle, wherein the secondary message transceiving unit 388 is configured to receive the obstacle presence message and transmit a third indication message to the two drive motors 382 to instruct the two wheels 384 to perform obstacle avoidance operations.

In the device of the self-propelled physiological fluid collection system of the present invention, the primary message transceiving unit 35 further comprises at least one weight information database configured to store weight information. The weight information includes a group selected from personal data, physiological fluid weight, time of weight sensing, and combinations thereof.

Through the device of the self-propelled physiological fluid collection system disclosed by the present invention, the design of the device, and the weight sensing unit 33, the sensor 34, the primary message transceiving unit 35, the tertiary driving unit 36, the container holder 37, and the mobility driving system 38 installed thereon can obtain and record a patient's physiological fluid weight in real-time and accurately monitor his physiological functions, thereby providing excellent medical and healthcare effectiveness.

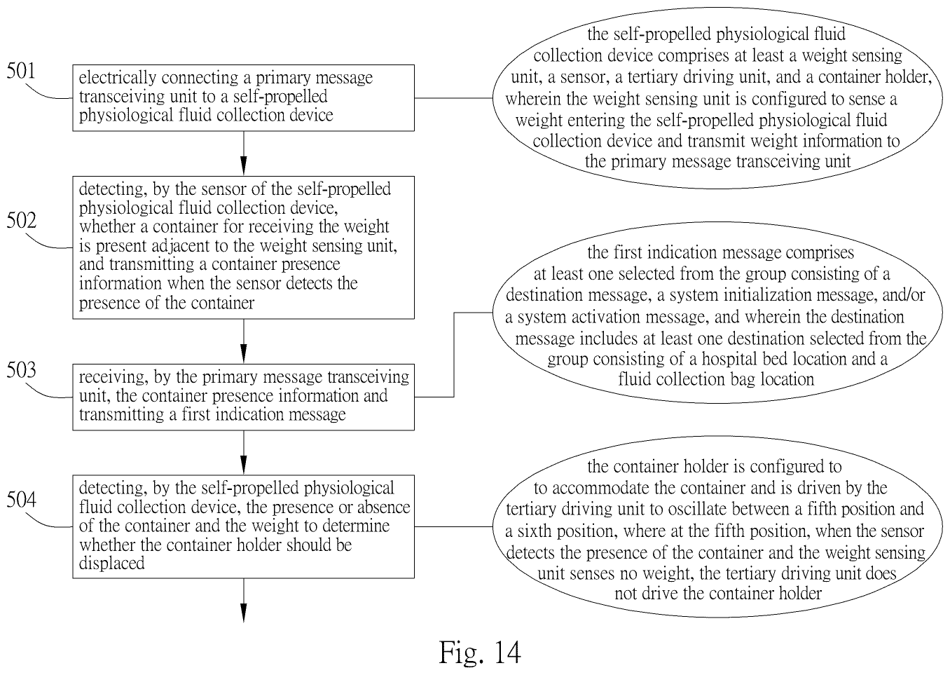

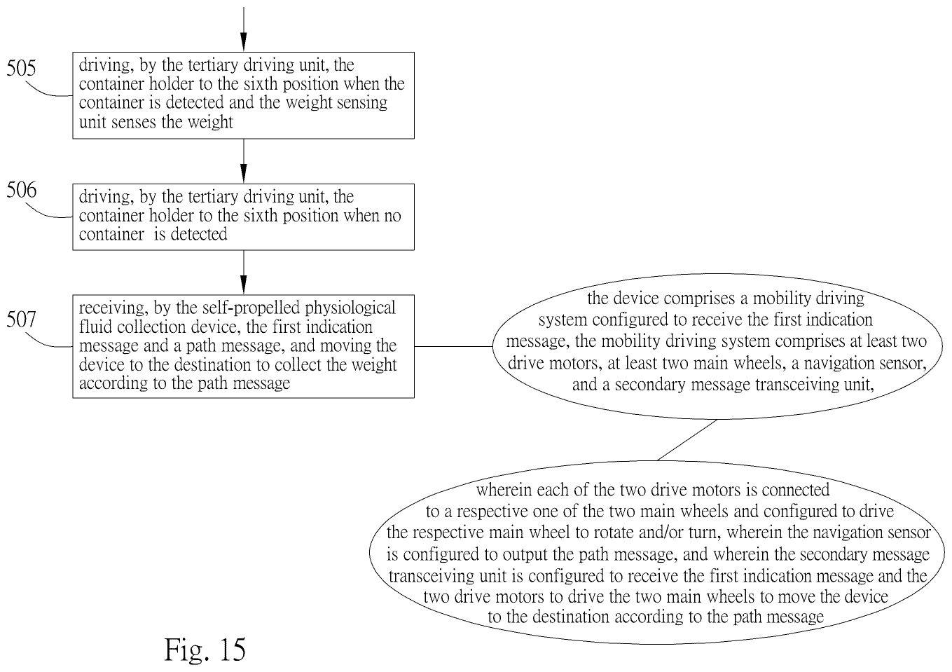

Referring to FIG. 14, the method of operating the self-propelled physiological fluid collection system comprises the following steps:
Step 501: electrically connecting a primary message transceiving unit 35 to a self-propelled physiological fluid collection device, wherein the self-propelled physiological fluid collection device comprises at least a weight sensing unit 33, a sensor 34, a tertiary driving unit 36, and a container holder 37, wherein the weight sensing unit 33 is configured to sense a weight entering the self-propelled physiological fluid collection device and transmit weight information to the primary message transceiving unit 35;
Step 502: detecting, by the sensor 34 of the self-propelled physiological fluid collection device, whether a container for receiving the weight is present adjacent to the weight sensing unit 33, and transmitting a container presence information when the sensor 34 detects the presence of the container;
Step 503: receiving, by the primary message transceiving unit 35, the container presence information and transmitting a first indication message, wherein the first indication message comprises at least one selected from the group consisting of a destination message, a system initialization message, and/or a system activation message, and wherein the destination message includes at least one destination selected from the group consisting of a hospital bed location and a fluid collection bag location;
Step 504: detecting, by the self-propelled physiological fluid collection device, the presence or absence of the container and the weight to determine whether the container holder 37 should be displaced, wherein the container holder 37 is configured to accommodate the container and is driven by the tertiary driving unit 36 to oscillate between a fifth position and a sixth position, where at the fifth position, when the sensor 34 detects the presence of the container and the weight sensing unit 33 senses no weight, the tertiary driving unit 36 does not drive the container holder 37;
Step 505: driving, by the tertiary driving unit 36, the container holder 37 to the sixth position when the container is detected and the weight sensing unit 33 senses the weight;
Step 506: driving, by the tertiary driving unit 36, the container holder 37 to the sixth position when no container is detected;
Step 507: receiving, by the self-propelled physiological fluid collection device, the first indication message and a path message, and moving the device to the destination to collect the weight according to the path message, wherein the device comprises a mobility driving system 38 configured to receive the first indication message, the mobility driving system 38 comprises at least two drive motors 382, at least two main wheels 384, a navigation sensor 386, and a secondary message transceiving unit 388, wherein each of the two drive motors 382 is connected to a respective one of the two main wheels 384 and configured to drive the respective main wheel 384 to rotate and/or turn, wherein the navigation sensor 386 is configured to output the path message, and wherein the secondary message transceiving unit 388 is configured to receive the first indication message and the path message and to transmit a second indication message to the two drive motors 382 to drive the two main wheels 384 to move the device to the destination according to the path message.

In the above method of the present invention, the navigation sensor 386 is a magnetic field sensor configured to output a magnetic field message and to sense a magnetic field path such as a magnetic strip path disposed on a ground surface where the device is configured to follow the magnetic field path for movement.

In the above method of the present invention, the navigation sensor 386 is an image sensor configured to output an image message to the secondary message transceiving unit 388, wherein the secondary message transceiving unit 388 is configured to transmit the second indication message to the two drive motors 382 to drive the two main wheels 384 to move the device.

The above method of the present invention further comprises steps of detecting, by the automated device, a presence or absence of an obstacle; transmitting an obstacle presence information to the secondary message transceiving unit 388; and transmitting, by the secondary message transceiving unit 388, a third indication message to the two drive motors 382 to instruct the two main wheels 384 to perform obstacle avoidance operations, wherein the device comprises an obstacle detection module 39 including at least three spaced-apart emitters selected from the group consisting of ultrasonic emitters and infrared emitters and disposed on the device, wherein the obstacle detection module 39 is configured to emit outward waves to detect the presence or absence of the obstacle, and transmit an obstacle presence message upon detecting the presence of the obstacle.

The above method of the present invention further comprises a step of storing the weight information in a weight information database, wherein the primary message transceiving unit 35 includes the weight information database, and the weight information includes at least one selected from the group consisting of personal data, physiological fluid weight, time of weight sensing, and combinations thereof.

In the above method of the present invention, the mobility driving system 38 further includes at least one auxiliary wheel 384′ configured to provide stable movement of the self-propelled physiological fluid collection device.

What is claimed is:

1. An automated oscillating rack, comprising:
a base having a first side surface attached to a support frame having a mounting space for accommodating a fluid collection bag;
a primary driving unit having a first side disposed on a second side surface of the base, wherein the second side surface is adjacent to the first side surface; and
an oscillating assembly comprising:
a main body having a first side disposed at a first end of the primary driving unit, wherein the primary driving unit drives the main body to move between a first position and a second position;
a flow channel formed as a passage and disposed on the main body, wherein a first end of the flow channel communicates with an outlet of the fluid collection bag;
a secondary driving unit disposed on a second side of the main body relative to the flow channel; and
a flow control switch having a first end attached to a middle section of the flow channel for opening or closing the passage of the flow channel and a second end disposed at a first end of the secondary driving unit, wherein the secondary driving unit drives the first end of the flow control switch to move between a third position and a fourth position, thereby causing the passage of the flow channel to be opened or closed.

2. The automated oscillating rack according to claim 1, wherein the main body undergoes an angular displacement between the first position and the second position, wherein when the main body is at the first position, a second end of the flow channel faces upward, and when the main body is at the second position, the second end of the flow channel faces downward.

3. The automated oscillating rack according to claim 1, wherein the flow channel is a flexible tube, and wherein the second end of the flow control switch corresponds to a rotation axis for rotation causing the first end of the flow control switch to undergo an angular displacement between the third position and the fourth position, wherein the first end of the flow control switch comprises either a cam or a cam block that deviates from the rotation axis in an axial direction, such that when the cam or the cam block is at the third position, the cam or the cam block does not compress the flow channel thereby opening the passage of the flow channel, and when the cam or the cam block is at the fourth position, the cam or the cam block compresses the flow channel thereby closing the passage of the flow channel.

4. The automated oscillating rack according to claim 1, further comprising an oscillation stop button disposed on the base and corresponding to the second position, wherein the oscillation stop button is electrically connected to the primary driving unit such that when the primary driving unit drives the main body to move between the first position and the second position, the main body presses the oscillation stop button, thereby causing the primary driving unit to stop.

5. The automated oscillating rack according to claim 2, further comprising an oscillation stop button disposed on the base and corresponding to the second position, wherein the oscillation stop button is electrically connected to the primary driving unit such that when the primary driving unit drives the main body to move between the first position and the second position, the main body presses the oscillation stop button, thereby causing the primary driving unit to stop.

6. The automated oscillating rack according to claim 1, further comprising a switch stopper including an extension rod extending outward from the middle section of the flow control switch by a predetermined length, and a first stop button and a second stop button disposed on the main body and positioned on opposite sides of the flow control switch, wherein the first stop button and the second stop button are electrically connected to the secondary driving unit and correspond to the third position and the fourth position, respectively, such that when the secondary driving unit drives the first end of the flow control switch to move between the third position and the fourth position, the switch stopper is configured to prevent the first stop button and the second stop button from being pressed simultaneously, thereby causing the secondary driving unit to stop.

7. The automated oscillating rack according to claim 2, further comprising a switch stopper including an extension rod extending outward from the middle section of the flow control switch by a predetermined length, and a first stop button and a second stop button disposed on the main body and positioned on opposite sides of the flow control switch, wherein the first stop button and the second stop button are electrically connected to the secondary driving unit and correspond to the third position and the fourth position, respectively, such that when the secondary driving unit drives the first end of the flow control switch to move between the third position and the fourth position, the switch stopper is configured to prevent the first stop button and the second stop button from being pressed simultaneously, thereby causing the secondary driving unit to stop.

8. The automated oscillating rack according to claim 1, further comprising a message transceiving unit electrically connected to a command transceiving unit, the primary driving unit, and the secondary driving unit, such that the command transceiving unit notifies the message transceiving unit to instruct the primary driving unit and the secondary driving unit to perform an oscillating motion to complete a physiological fluid collection operation.

9. The automated oscillating rack according to claim 2, further comprising a message transceiving unit electrically connected to a command transceiving unit, the primary driving unit, and the secondary driving unit, such that the command transceiving unit notifies the message transceiving unit to instruct the primary driving unit and the secondary driving unit to perform an oscillating motion to complete a physiological fluid collection operation.

10. A method for operating a self-propelled physiological fluid collection system, comprising:
(a) a step of electrically connecting a primary message transceiving unit to a self-propelled physiological fluid collection device; wherein,
the self-propelled physiological fluid collection device comprises at least a weight sensing unit, a sensor, a tertiary driving unit, and a container holder; and
the weight sensing unit is configured to sense a weight entering the self-propelled physiological fluid collection device and transmit weight information;
(b) a step of detecting, by the sensor of the self-propelled physiological fluid collection device, the presence of a container for receiving the weight adjacent to the weight sensing unit and transmitting a container presence information;
(c) a step of receiving, by the primary message transceiving unit, the container presence information and transmitting a first indication message; wherein,
the first indication message comprises at least one destination;
(d) a step of detecting, by the self-propelled physiological fluid collection device, the presence or absence of the container and the weight to determine whether the container holder should be displaced; wherein,
the container holder is configured to accommodate the container and is driven by the tertiary driving unit to oscillate between a fifth position and a sixth position, where at the fifth position, when the sensor detects the presence of the container and the weight sensing unit senses no weight, the tertiary driving unit does not drive the container holder;
(e) a step of driving, by the tertiary driving unit, the container holder to the sixth position when the container is detected and the weight sensing unit senses the weight;
(f) a step of driving, by the tertiary driving unit, the container holder to the sixth position when no container is detected; and
(g) a step of receiving, by the self-propelled physiological fluid collection device, the first indication message and a path message, and moving the self-propelled physiological fluid collection device to the destination to collect the weight according to the path message; wherein,
the self-propelled physiological fluid collection device comprises a mobility driving system configured to receive the first indication message and to drive the self-propelled physiological fluid collection device, and the mobility driving system comprises a navigation sensor and a secondary message transceiving unit, wherein the navigation sensor is configured to output the path message, and the secondary message transceiving unit is configured to receive the first indication message and the path message and to transmit a second indication message to the mobility driving system to move the self-propelled physiological fluid collection device to the destination according to the path message.

11. The method according to claim 10, wherein the weight sensing unit is disposed on the self-propelled physiological fluid collection device and is configured to transmit the weight information to the primary message transceiving unit.

12. The method according to claim 10, wherein the first indication message comprises a system initialization message and/or a system activation message.

13. The method according to claim 10, further comprising steps of detecting, by the self-propelled physiological fluid collection device, a presence or absence of an obstacle; transmitting an obstacle presence information to the secondary message transceiving unit; and transmitting, by the secondary message transceiving unit, a third indication message to the two drive motors to instruct the two main wheels to perform obstacle avoidance operations, wherein an obstacle detection module is disposed on the self-propelled physiological fluid collection device and is configured to emit outward waves to detect the presence or absence of the obstacle and to transmit an obstacle presence message upon detecting the presence of the obstacle.

14. The method according to claim 10, further comprising a step of storing the weight information in a weight information database, wherein the primary message transceiving unit includes the weight information database, and the weight information includes at least one selected from the group consisting of personal data, physiological fluid weight, time of weight sensing, and combinations thereof.

15. The method according to claim 10, wherein the primary message transceiving unit comprises a computer, and the secondary message transceiving unit comprises at least one application installed on a circuit board.

16. The method according to claim 11, wherein the primary message transceiving unit comprises a computer, and the secondary message transceiving unit comprises at least one application installed on a circuit board.

17. The method according to claim 12, wherein the primary message transceiving unit comprises a computer, and the secondary message transceiving unit comprises at least one application installed on a circuit board.

18. The method according to claim 13, wherein the primary message transceiving unit comprises a computer, and the secondary message transceiving unit comprises at least one application installed on a circuit board.

19. The method according to claim 14, wherein the primary message transceiving unit comprises a computer, and the secondary message transceiving unit comprises at least one application installed on a circuit board.

20. The method according to claim 10, wherein the weight is physiological fluid from a fluid collection bag.

21. The method according to claim 11, wherein the weight is physiological fluid from a fluid collection bag.

22. The method according to claim 12, wherein the weight is physiological fluid from a fluid collection bag.

23. The method according to claim 13, wherein the weight is physiological fluid from a fluid collection bag.

24. The method according to claim 14, wherein the weight is physiological fluid from a fluid collection bag.

25. A self-propelled physiological fluid collection device according to claim 10, comprising at least:
a collection unit comprising a bottom surface and a peripheral side surface surrounding a periphery of the bottom surface to form a recessed enclosure having at least one opening;
a weight sensing unit comprising a side disposed on an inner side of the bottom surface and other side configured to sense a weight containing a physiological fluid placed through the opening and to transmit weight information corresponding to the sensed weight;
a sensor disposed on the collection unit and configured to detect presence or absence of the container for receiving the weight adjacent to the weight sensing unit and transmit container presence information;
a primary message transceiving unit electrically connected to the weight sensing unit and the sensor and configured to receive the weight information and the container presence information and to transmit a first indication message comprising at least one destination message;
a tertiary driving unit disposed on the inner side of the bottom surface;
a side of a container holder disposed on a side of the tertiary driving unit, configured to accommodate the container, and driven by the tertiary driving unit to oscillate between a fifth position and a sixth position, when the sensor detects the presence of the container and the weight sensing unit detects the absence of the weight at the fifth position, the tertiary driving unit maintains the container holder at the fifth position; wherein,
(1) when the sensor detects the presence of the container and the weight sensing unit detects the absence of the weight, the tertiary driving unit drives the container holder to the sixth position;
(2) when the sensor detects the absence of the container, the tertiary driving unit drives the container holder to the sixth position; and
a mobility driving system having a side disposed on an outer side of the bottom surface and electrically connected to the primary message transceiving unit configured to receive the first indication message, wherein the mobility driving system includes a navigation sensor configured to output a path message and a secondary message transceiving unit configured to receive the first indication message and the path message and to transmit a second indication message to the mobility driving system to move the collection unit to the destination according to the path message for collecting the weight.

26. The device according to claim 25, wherein the primary message transceiving unit further comprises at least one weight information database configured to store weight information, wherein the weight information includes at least one selected from the group consisting of personal data, physiological fluid weight, time of weight sensing, and combinations thereof.

27. The device according to claim 25, wherein the primary message transceiving unit comprises a computer, and the secondary message transceiving unit comprises at least one application installed on a circuit board.

28. The device according to claim 26, wherein the primary message transceiving unit comprises a computer, and the secondary message transceiving unit comprises at least one application installed on a circuit board.

29. The device according to claim 25, wherein the weight is physiological fluid from a fluid collection bag.

30. The device according to claim 26, wherein the weight is physiological fluid from a fluid collection bag.
